(19) United States
(12) Patent Application Publication
(10) Pub. No.: US 2005/0131246 A1
(43) Pub. Date: Jun. 16, 2005

(54) Method for producing 2,2-dichloro or dibromo-phenyl alkyl acetates

(75) Inventors: Sylvia Krich, Altenber bei Linz (AT); Bernhard Holub, Strabham (AT)

Correspondence Address: WENDEROTH, LIND & PONACK, L.L.P., 2033 K STREET N. W., SUITE 800, WASHINGTON, DC, 20006-1021, US

(73) Assignee: DSM Fine Chemicals Austria Nfg GmbH & Co KG, Linz (AT)

(21) Appl. No.: 10/507,305

(22) PCT Filed: Mar. 10, 2003
(86) PCT No.: PCT/EP03/02407

(30) Foreign Application Priority Data
Apr. 4, 2002 (AT) A 523/2002

(51) Int. Cl. C07C 69/76

(52) U.S. Cl. 560/105

(57) ABSTRACT

The invention relates to an improved method for producing 2,2-dichloro or dibromo-phenyl alkyl acetates of formula (1), in which X represents Cl or Br, n represents a whole number from 1 to 5, R represents hydrogen, C₁-C₈ alkyl, aryl, heteroaryl, C₁-C₈ alkoxy, aryloxy or halogen and R1 represents C₁-C₈ alkyl. According to said method, a 2,2-dichloro- or dibromo-phenylacetonitrile of formula (II) is reacted in 0.8 to 2 mol water per mol nitrile of formula (II), 1 to 8 mol alcohol of formula R1OH (III) per mol nitrile of formula (II) and in the presence of 1 to 3 mol HCl or HBr per mol nitrile of formula (II), optionally in the presence of a solvent that is inert in the reaction conditions, to form the corresponding 2,2-dichloro- or dibromo-phenyl alkyl acetates of formula (I), whereby the reaction temperature in the first phase lies between 30 and 60° C. and in the second phase between 60 and 100° C. Once the reaction has taken place, the reaction mixture is cooled to between 20 and 40° C., diluted with water and the 2,2-dichloro- or dibromo-phenyl alkyl acetate of formula (I) is isolated.

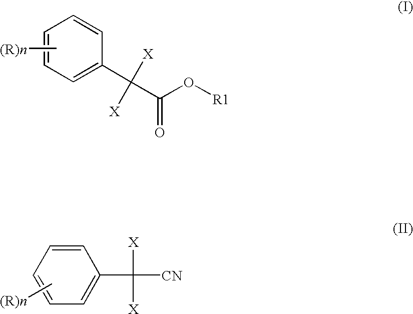

Alkyl 2,2-dichloro- or dibromophenylacetates are used as intermediates, for example, in the preparation of pesticides or as vulcanization accelerants for elastomers.

These compounds-were initially prepared, as described, for example, in EP 0 075 356, by reaction of phosphorus pentachloride with a phenylglyoxylic ester which had been obtained from benzoyl cyanide. Since benzoyl cyanide was a very expensive reactant, benzoyl cyanide was used, in an alternative method, as a reactant for the preparation of 2,2-dichlorophenylacetonitrile, for example according to EP 0 518 412, which is then converted to the desired alkyl 2,2-dichlorophenylacetate according to EP 0 075 356.

In this conversion, 2,2-dichlorophenylacetonitrile is reacted with water and an alcohol in the presence of a hydrogen halide, preferably gaseous HCl, at a temperature of from 0 to 80° C., preferably from 15 to 50° C.

A problem in the procedure described in EP 0 075 356 is the formation of the 2,2-dichlorphenylacetamide by-product which then has to be removed from the reaction mixture and distinctly reduces the yield of the desired end compound. A further problem is, when ethanol as the alcohol and HCl are used, the formation of ethyl chloride, a poisonous, inflammable by-product which must not be emitted. Further side reactions are the hydrolysis of the end product to give the corresponding phenylglyoxylic ester or to give phenylglyoxylic acid. However, in order to satisfy the specifications for further processing, these by-products, for instance phenylglyoxylic acid, must only be present in the end product in extremely small amounts.

In the preparation of alkyl 2,2-dichlorophenylacetates under the parameters and process sequence described in EP 0 075 356, the desired end products is additionally obtained in yields of only up to 75% and with high proportions of highly differing by-products.

It is accordingly an object of the present invention to find an improved process for preparing alkyl 2,2-dichloro- or dibromophenylacetates, which makes the desired products obtainable in higher yields and with higher purity.

The invention accordingly provides an improved process for preparing alkyl 2,2-dichloro- or dibromophenylacetates of the formula 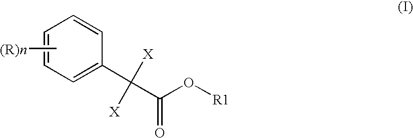
in which X is Cl or Br, n may be an integer from 1 to 5, R is hydrogen, C₁-C₈-alkyl, aryl, heteroaryl, C₁-C₈-alkoxy, aryloxy or halogen, and R1 is C₁-C₈-alkyl, characterized in that a 2,2-dichloro- or dibromophenylacetonitrile of the formula 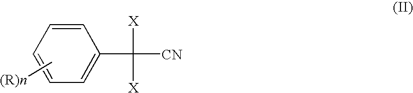
in which X, n and R are each as defined above, in from 0.8 to 2 mol of water per mole of nitrile of the formula (II), from 1 to 8 mol of alcohol of the formula
R1OH (III)
in which R1 is as defined above, per mole of nitrile of the formula (II) and in the presence of from 1 to 3 mol of HCl or HBr per mole of nitrile of the formula (II), optionally in the presence of a solvent inert under the reaction conditions, is converted to the corresponding alkyl 2,2-dichloro- or dibromophenylacetate of the formula (I), the reaction temperature in the first phase of the conversion being from 30 to 60° C. and, in the second phase, from 60 to 100° C., whereupon, on completion of conversion, the reaction mixture is cooled to from 20 to 40° C. and diluted with water, and the corresponding alkyl 2,2-dichloro- or dibromophenylacetate of the formula (I) is isolated.

In the process according to the invention, a 2,2-dichloro- or dibromophenylacetonitrile of the formula (II) is reacted with water, alcohol R1OH and HCl or HBr, optionally in the presence of a solvent inert under the reaction conditions to give the corresponding alkyl 2,2-dichloro- or dibromophenylacetate of the formula (I).

In the formula (II), X is chlorine or bromine, preferably chlorine.

n is an integer from 1 to 5.

R may be hydrogen, C₁-C₈-alkyl, aryl, heteroaryl, C₁-C₈-alkoxy, aryloxy or halogen.

C₁-C₈-Alkyl refers to linear, branched or cyclic alkyl radicals having from 1 to 8 carbon atoms, such as methyl, ethyl, isopropyl, n-propyl, cyclopropyl, n-butyl, tert-butyl, n-propyl, cyclopropyl, hexyl or octyl.

Preference is given to linear or branched C₁-C₄-alkyl radicals.

C₁-C₈-Alkoxy refers to alkoxy radicals having from 1 to 8 carbon atoms in which the alkyl moiety may be linear or branched, such as methyloxy, ethyloxy, isopropyloxy, n-propyloxy, n-butyloxy, tert-butyloxy, n-propyloxy, hexyloxy or octyloxy.

Preference is given to linear or branched C₁-C₄-alkoxy radicals.

The alkyl or the alkoxy group may optionally be mono- or polysubstituted by groups inert under the reaction conditions, such as optionally substituted aryl or heteroaryl groups, halogen, alkoxy, aryloxy, etc.

Aryl and aryloxy are preferably aromatic radicals having from 6 to 20 carbon atoms, such as phenyl, biphenyl, naphthyl, indenyl, fluorenyl, phenoxy, etc.

Preferred aromatic radicals are phenyl and phenoxy.

The aryl group may optionally be mono- or polysubstituted by groups inert under the reaction conditions, such as optionally substituted aryl or heteroaryl groups, halogen, alkoxy, aryloxy, etc.

However, the aryl group may also be fused to the phenyl ring, so that the phenyl ring and R as aryl form an optionally substituted, fused aromatic ring system, such as indenyl, fluorenyl, naphthyl, etc.

Heteroaryl refers to aromatic radicals which contain at least one sulfur, oxygen or nitrogen atom in the ring or ring system. These are, for example, furyl, pyridyl, pyrimidyl, thienyl, isothiazolyl, imidazolyl, quinolyl, benzothienyl, indolyl, pyrrolyl, etc.

The heteroaryl group may optionally be mono- or polysubstituted by groups inert under the reaction conditions, such as optionally substituted aryl or heteroaryl groups, halogen, alkoxy, aryloxy, etc.

However, the heteroaryl group may also be fused to the phenyl ring, so that the phenyl ring and R as heteroaryl form an optionally substituted, fused ring system, such as quinolinyl, indolyl, isoindolyl, coumaronyl, phthalizinyl, etc.

Halogen refers to fluorine, chlorine, bromine and iodine, and preference is given to fluorine, bromine and chlorine.

If R is not hydrogen, n is preferably an integer from 1 to 3, more preferably 1 or 2.

R is preferably hydrogen, an unsubstituted, linear or branched C₁-C₄-alkyl or alkoxy radical, unsubstituted phenyl or phenoxy, or chlorine. R is more preferably hydrogen.

The reactants of the formula (II) used for the process according to the invention are commercially available or can be prepared, for example, from benzyl cyanide, for instance according to EP 0 518 412.

According to the invention, the nitrites of the formula (II) are reacted with from 0.8 to 2 mol of water per mole of nitrile of the formula (II), from 1 to 8 mol of alcohol of the formula R1OH (III) per mole of nitrile of the formula (II) and from 1 to 3 mol of HCl or HBr per mole of nitrile of the formula (II).

Suitable alcohols of the formula (III) are those in which R1 is C₁-C₈-alkyl. Examples thereof are methanol, ethanol, n-propanol, isopropanol, isobutanol, n-pentanol or n-hexanol.

The selection of the alcohol depends upon the desired ester in the end product. The alcohol of the formula (III) used is preferably methanol, ethanol or n-butanol, more preferably ethanol.

The alcohol of the formula (III) is used in an amount of from 1 to 8 mol per mole of nitrile of the formula (II), preferably from 3 to 5 mol per mole of nitrile of the formula (II). If desired, larger amounts of alcohol may also be used, but are not viable for economic reasons.

When the alcohol is used in an amount of from 1 to about 3 mol per mole of nitrile, it is advantageous to use an additional solvent inert under the reaction conditions. Suitable solvents are, for example, ethers such as methyl tert-butyl ether (MTBE), diethyl ether, tetrahydrofuran (THF), dioxane or higher ethers such as ethylene glycol dimethyl ether, etc., or optionally halogenated hydrocarbons such as toluene, hexane, heptane, dichloromethane, chlorobenzene, etc.

Water is added to the reaction mixture in an amount of from 0.8 to 2 mol per mole of nitrile of the formula (II), preferably from 0.9 to 1.5 mol per mole of nitrile of the formula (II). Additionally, from 1 to 3 mol of HCl or HBr per mole of nitrile of the formula (II) are added.

In the process according to the invention, the nitrile of the formula (II) may first be dissolved in the desired alcohol of the formula (III), optionally an solvent inert under the reaction conditions and in water, whereupon gaseous HCl or HBr is subsequently introduced. The gaseous HCl or HBr introduced may also be the HCl or HBr offgas obtained in the chlorination with chlorine gas, or bromination with Br₂, of optionally substituted benzyl cyanide to give the desired nitriles of the formula (II), for example according to EP 0 518 412, which allows direct coupling of the preparation of the nitrile reactant of the formula (II) to the preparation of the desired alkyl dichloro- or dibromophenylacetate of the formula (I) to be achieved.

The invention accordingly further provides a process for preparing alkyl 2,2-dichloro- or dibromophenylacetates of the formula (I), characterized in that, in a 1st stage, an optionally substituted benzyl cyanide of the formula 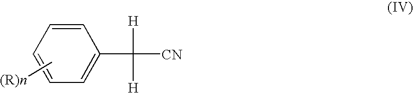
is reacted with chlorine in the presence of catalytic amounts of hydrogen chloride gas, or with a brominating agent, to give the corresponding nitrile of the formula (II) and the HCl or HBr offgas which forms is used in the second stage to convert the nitrile of the formula (II) to the corresponding alkyl 2,2-dichloro- or dibromophenylacetate of the formula (I).

Stage 1 may be carried out in a similar manner to EP 0 518 412.

In a preferred variant, an alcohol/water/HCl or HBr mixture is used for the conversion of the nitrile of the formula (II).

This three-substance mixture may be obtained by introducing hydrogen chloride gas or HBr into a mixture of water and alcohol.

Particular preference is given to obtaining the alcohol/water/HCl or HBr mixture by passing the HCl or HBr offgas obtained in the halogenation with Cl₂ or Br₂ of optionally substituted benzyl cyanides to give the desired nitriles of the formula (II), for example according to EP 0 518 412, into a mixture of water and alcohol. This has the advantage that the preparation of the nitrile of the formula (II) does not have to be coupled directly with the preparation of the alkyl dichloro- or dibromophenylacetate of the formula (I). HCl or HBr which are obtained as the offgas from the halogenation may thus also be intermediately stored in the form of an aqueous, alcoholic solution.

However, the mixture may also be obtained by passing HCl or HBr, or HCl or HBr offgas, into a solution of alcohol and aqueous HCl or HBr, or into alcohol with subsequent dilution with water.

The desired molar ratio in the three-substance mixture may optionally be adjusted by diluting the aqueous, alcoholic HCl or HBr solution present with alcohol and/or water.

When an alcohol/water/HCl or HBr mixture is used in the process according to the invention, it is possible first to initially charge the nitrile of the formula (II) and subsequently to meter in the three-substance mixture. However, it is also possible to initially charge the three-substance mixture and then add the nitrile of the formula (II).

In the process according to the invention, the conversion in the first phase is effected at a temperature of from 30 to 60° C., preferably from 35 to 55° C.

To this end, for example, either the nitrile of the formula (II) or the three-substance mixture is initially charged and heated from to 30 to 60° C., and the missing reaction component is subsequently metered in at this temperature. The thus obtained reaction mixture is then stirred at this temperature for a further few minutes up to several hours, preferably from 30 minutes up to 5 hours.

In the second phase, the reaction mixture is heated to from 60 to 100° C., preferably to from 65 to 80° C., and again stirred at this temperature for a further few minutes up to several hours, preferably from 30 minutes up to 10 hours.

After checking for complete conversion, the reaction mixture is cooled to from 20 to 40° C. and sufficient water is added that the precipitated ammonium chloride or bromide is just dissolved and a phase separation occurs. Subsequently, the aqueous phase may, if desired, be extracted by means of customary extractants, such as hexane, heptane, toluene, ethers or esters. The extract is then combined with the organic phase.

The end product can then be isolated, for example, by initially distilling off any extractant, water and alcohol at atmospheric pressure and a max. temperature of 90° C., and then, under reduced pressure, low boilers and the by-products, phenylglyoxylic acid, ethyl phenylglyoxylate, ethyl phenylacetate (from incomplete halogenation) and monochloro- or monobromophenylacetic esters, until a constant boiling temperature is attained, so that the appropriate end product of the formula (I) remains in the residue. For further purification, the product may be distilled overhead.

However, after combining the organic phases, the water may also first be removed on a water separator and then alcohol and any extractant distilled off at atmospheric pressure.

When the crude end product still contains too many organic acids which are formed by the hydrolysis of the product, the crude product is admixed once again with one of the extractants cited above and alcohol, and worked up again distillatively, in the course of which reesterification is effected.

The process according to the invention provides alkyl 2,2-dichloro- or dibromophenylacetates of the formula (I) in higher yields and higher purities compared to the prior art, in the course of which substantially fewer emissions are formed than in processes customary hitherto and the offgas utilization additionally results in fewer raw materials being required.

### EXAMPLE 1

### Laboratory Experiment, Chlorination of Benzyl Cyanide to 2,2-dichlorophenylacetonitrile

1436 g (12.27 mol) of benzyl cyanide were charged into an enamel autoclave which had been inertized beforehand. Afterward, 239 g (6.55 mol=0.533 eq.) of hydrogen chloride gas were introduced with the offgas valve open, then the offgas valve was closed and the autoclave heated to 40° C.

Subsequently, 1830 g (25.77 mol=2.10 eq.) of chlorine were introduced at 60-65° C. and 3 bar over 6 hours. The temperature rose rapidly within the first few minutes, but then remained approximately constant as a function of the introduction rate. On attainment of an internal pressure of 3 bar, the offgas valve was opened slightly, so that the pressure remained constant at 3 bar.

Toward the end of the reaction, temperature and pressure fell slightly. On completion of chlorine introduction, the offgas valve was closed and the mixture stirred at 55° C. for 30 minutes.

Afterward, the autoclave was decompressed, and nitrogen was passed through to drive out chlorine and HCl gas.

The offgas scrubber was charged with 2823 g (61.3 mol=5 eq.) of ethanol and 340 g of concentrated hydrochloric acid (12.27 mol=1 eq. corresponding to 221 g of water and 3.26 mol=0.266 eq.=119 g of hydrochloric acid) and 95% of the offgas from the chlorination (as long as no chlorine (max. 3%) had been in the offgas) was introduced at 10-15° C. Connected downstream was a safety scrubber (charged with 10% sodium hydroxide solution).

The remaining offgas was introduced into 2 scrubbers connected in series and having 10% sodium hydroxide solution.

Yield:

2200 g of 2,2-dichlorophenylacetonitrile (96.3% of theory)

3950 g of 24.5% ethanolic hydrochloric acid (yield of HCl from the chlorination: 73% of theory).

### EXAMPLE 2

### Preparation of Ethyl 2,2-dichlorophenylacetate from 2,2-dichlorophenylacetonitrile (2nd Stage, Laboratory (Variant I))

70.0 g (0.38 mol) of distilled 2,2-dichlorophenylacetonitrile, prepared as in Example 1, were heated to 40° C. 123.57 g of ethanolic hydrochloric acid obtained from a chlorination similarly to Example 1 (30.22 g=0.83 mol corresponding to 2.18 eq. of hydrochloric acid, 6.77 g=3.76 mol=0.99 eq. of water and 86.6 g=1.88 mol=4.95 eq. of ethanol) were then metered in at 40° C. within 30 minutes, and, on completion of addition, the mixture was stirred at 40° C. for a further 2 hours. The mixture was then heated to 75° C. and stirred for a further 3 hours. After checking for complete conversion, the reaction mixture was cooled to 30° C. and 115 g of water were added.

The mixture was stirred until the solid (ammonium chloride) had dissolved fully. Afterward, the organic phase was removed and the remaining aqueous phase extracted with 18 g of hexane. The organic extract was combined with the product phase obtained beforehand and distilled initially at atmospheric pressure to remove ethanol, water and hexane. Subsequently, incipient distillation was effected at 10 mbar until a constant boiling temperature of 128° C. had been attained.

The remaining residue contained 97.8% w/w of ethyl 2,2-dichlorophenylacetate.

Yield: 83.6 g of ethyl 2,2-dichlorophenylacetate (95% of theory), 97.8% w/w.

### EXAMPLE 3

### Preparation of Ethyl 2,2-dichlorophenylacetate from 2,2-dichlorophenylacetonitrile (2nd Stage, Laboratory (Variant II))

123.57 g of ethanolic hydrochloric acid obtained from a chlorination similarly to Example 1 (30.22 g=0.83 mol=2.18 eq. of hydrochloric acid, 6.77 g=3.76 mol=0.99 eq. of water; 86.6 g=1.88 mol=4.95 eq. of ethanol) were heated to 40° C.

70.0 g (0.38 mol) of 2,2-dichlorophenylacetonitrile were then metered in within 30 minutes and, on completion of addition, the mixture was stirred at 40° C. for a further 2 hours. Afterward, the mixture was heated to 75° C. and stirred for a further 3 hours. After checking for complete conversion, the reaction mixture was cooled to 30° C. and 115 g of water were added.

The mixture was stirred until the solid (ammonium chloride) had dissolved fully. Afterward, the organic phase was removed and the remaining aqueous phase extracted using 18 g of hexane. The organic extract was combined with the product phase obtained beforehand and the water was initially removed on a water separator. Afterward, the mixture was distilled at atmospheric pressure to remove ethanol and hexane. Subsequently, incipient distillation was effected at 10 mbar until a constant boiling temperature of 128° C. had been attained.

The remaining residue contained 93.9% w/w of ethyl 2,2-dichlorophenylacetate. For further purification, the product was distilled overhead.

Yield: 70 g of ethyl 2,2-dichlorophenylacetate (80% of theory), 99.4% w/w.

### EXAMPLE 4

### Pilot Experiment, Chlorination of Benzyl Cyanide to 2,2-dichlorophenylacetonitrile

400 kg (3385 mol) of benzyl cyanide were charged into an enamel tank which had been inertized beforehand, then 40 kg (1096 mol=0.324 eq.) of hydrogen chloride gas were introduced with the offgas valve open at 40-50° C. over one hour, and then the offgas valve was closed.

Subsequently, 487 kg (6859 mol=2.026 eq.) of chlorine were introduced at 55-60° C. and 3-3.5 bar over 15 hours.

On completion of chlorine introduction, the offgas valve was closed and the mixture was stirred at 60-63° C. for 3 hours. Afterward, the tank was decompressed, and nitrogen passed through to drive out chlorine and HCl gas.

The offgas scrubber was charged with 475 kg (10326 mol=3.05 eq.) of ethanol and 94 kg of concentrated hydrochloric acid (3394 mol=1 eq.=61.1 kg of water and 901 mol=0.266 eq.=32.9 kg of hydrochloric acid) and the offgas from the chlorination was introduced at 10-15° C. up to a max. chlorine concentration of 3% in the offgas. Downstream was connected a safety scrubber (charged with 10% sodium hydroxide solution).

The remaining offgas was introduced directly into the above-described sodium hydroxide solution scrubber.

Yield:

636 kg of 2,2-dichlorophenylacetonitrile (˜theoretical yield)

825 kg of 35.2% ethanolic hydrochloric acid (yield of HCl from the chlorination: 89% of theory).

### EXAMPLE 5

### Preparation of Ethyl 2,2-dichlorophenylacetate from 2,2-dichlorophenylacetonitrile (2nd Stage, Pilot (Variant II))

410 kg of the ethanolic hydrochloric acid obtained in Example 4 (143.5 kg=3932 mol=2.31 eq. of hydrochloric acid; 30.5 kg=1694 mol=1 eq. of water; 236 kg=5130 mol=3.02 eq. of ethanol) were diluted with 100 kg (2174 mol=1.28 eq.) of ethanol and heated to 40° C.

316 kg (1699 mol) of 2,2-dichlorophenylacetonitrile were then metered in at 40° C. within 2 hours and, on completion of addition, the mixture was stirred at 40° C. for a further hour. Afterward, the mixture was heated to 70° C. and stirred for a further 6 hours. After checking for complete conversion, the reaction mixture was cooled to 30° C.

The resulting suspension was introduced into 570 l of water and stirred until the solid (ammonium chloride) had dissolved fully. Afterward, the organic phase was removed and the remaining aqueous phase extracted with 80 kg of hexane. The organic extract was combined with the product phase obtained beforehand and initially incipiently distilled at atmospheric pressure up to 90° C. (to remove ethanol, water and hexane) and subsequently fractionated at 7 mbar up to 135° C.

Yield: 325 kg of ethyl 2,2-dichlorophenylacetate (82% of theory), 98.8% w/w.

### EXAMPLE 6

### Preparation of Ethyl 2,2-dichlorophenylacetate from 2,2-dichlorophenylacetonitrile (2nd Stage, Pilot (Variant I))

325.3 kg (1749 mol) of 2,2-dichlorophenylacetonitrile were diluted with 100 kg (2174 mol=1.28 eq.) of ethanol and heated to 40° C.

410 kg of the ethanolic hydrochloric acid obtained in Example 4 (143.5 kg=3932 mol=2.25 eq. of hydrochloric acid; 30.5 kg=1694 mol=0.97 eq. of water; 236 kg=5130 mol=2.93 eq. of ethanol) were then metered in at 40° C. within 3 hours and, on completion of addition, the mixture was stirred at 40° C. for a further 3 hours. Afterward, the mixture was heated to 70° C. and stirred for a further 6 hours. After checking for complete conversion, the reaction mixture was cooled to 30° C.

The resulting suspension was introduced into 570 l of water and stirred until the solid (ammonium chloride) had dissolved fully. Subsequently, the organic phase was removed and the remaining aqueous phase extracted with 80 kg of hexane. The organic extract was combined with the product phase obtained beforehand and initially incipiently distilled at 20-25° C. and slightly reduced pressure (to remove ethanol, water and hexane) and subsequently fractionated at 7 mbar up to 135° C.

Yield: 346.4 kg of ethyl 2,2-dichlorophenylacetate (85% of theory), 98.9% w/w.

### EXAMPLE 7

### Preparation of Ethyl 2,2-dichlorophenylacetate from 2,2-dichlorophenylacetonitrile (2nd Stage, Pilot (Variant II with Reesterification))

375 kg of the ethanolic hydrochloric acid obtained in the chlorination of Example 4 (136.7 kg=3745 mol=2.16 eq.) of hydrochloric acid; 28.2 kg=1567.5 mol=0.91 eq. of water; 210.1 kg=4567 mol=2.64 eq. of ethanol) were diluted with 100 kg (2174 mol=1.26 eq.) of ethanol and heated to 40° C. 322 kg (1732 mol) of 2,2-dichlorophenylacetonitrile were then metered in at 40° C. within 2 hours and, on completion of addition, the mixture was stirred at 40° C. for a further hour. Afterward, the mixture was heated to 70° C. and stirred for a further 6 hours. After checking for complete conversion, the reaction mixture was cooled to 30° C.

The resulting suspension was introduced into 570 l of water and stirred until the solid (ammonium chloride) had dissolved fully. Subsequently, the organic phase was removed and the remaining aqueous phase extracted with 80 kg of hexane. The organic extract was combined with the product phase obtained beforehand and initially incipiently distilled at atmospheric pressure up to 120° C. (to remove ethanol, water and hexane) Since the crude product contained too many organic acids (from hydrolysis of the product), 40 kg of hexane and 20 kg of ethanol were added and distillation was again effected at atmospheric pressure up to 120° C. Subsequently, the crude product was fractionated at 7 mbar up to 135° C.

Yield: 329 kg of ethyl 2,2-dichlorophenylacetate (81.5% of theory), 98.0% w/w.

### EXAMPLE 8

### Reesterification, Laboratory

11.3 g of ethyl 2,2-dichlorophenylacetate having a content of 0.13% w/w of phenylglyoxylic acid (a hydrolysis product of ethyl 2,2-dichlorophenylacetate) was heated with 2 ml of hexane and 0.5 ml of ethanol at 70° C. for 2 hours. Afterward, the solvents were distilled off.

Result: The yield of phenylglyoxylic acid falls to 0.01% w/w

1. An improved process for preparing alkyl 2,2-dichloro- or dibromophenylacetates of the formula 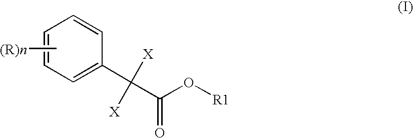
in which X is Cl or Br, n may be an integer from 1 to 5, R is hydrogen, C₁-C₈-alkyl, aryl, heteroaryl, C₁-C₈-alkoxy, aryloxy or halogen, and R1 is C₁-C₈-alkyl, characterized in that a 2,2-dichloro- or dibromophenylacetonitrile of the formula 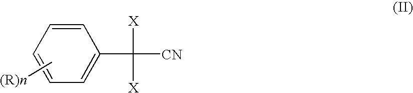
in which X, n and R are each as defined above, in from 0.8 to 2 mol of water per mole of nitrile of the formula (II), from 1 to 8 mol of alcohol of the formula
R1OH (III)
in which R1 is as defined above, per mole of nitrile of the formula (II) and
in the presence of from 1 to 3 mol of HCl or HBr per mole of nitrile of the formula (II), optionally in the presence of a solvent inert under the reaction conditions, is converted to the corresponding alkyl 2,2-dichloro- or dibromophenylacetate of the formula (I), the reaction temperature in the first phase of the conversion being from 30 to 60° C. and, in the second phase, from 60 to 100° C., whereupon, on completion of conversion, the reaction mixture is cooled to from 20 to 40° C. and diluted with water, and the corresponding alkyl 2,2-dichloro- or dibromophenylacetate of the formula (I) is isolated.

2. The process of claim 1, characterized in that, in the formula (I), R is hydrogen, C₁-C₄-alkyl, C₁-C₄-alkoxy, phenyl, phenoxy, fluorine, bromine or iodine, and n, if R is not hydrogen, is an integer from 1 to 3.

3. The process of claim 1, characterized in that the alcohol of the formula (III) used is methanol, ethanol or n-butanol.

4. The process of claim 1, characterized in that, in the case that the alcohol of the formula (II) is used in an amount of from 1 up to 3 mol per mole of nitrile of the formula (II), the reaction is carried out in the presence of a solvent, inert under the reaction conditions, from the group of methyl tert-butyl ether, diethyl ether, tetrahydrofuran, dioxane, ethylene glycol dimethyl ether, toluene, hexane, heptane, dichloromethane or chlorobenzene.

5. The process of claim 1, characterized in that the HCl or HBr used is the HCl or HBr offgas which is obtained in the reaction of optionally substituted benzene cyanide of the formula 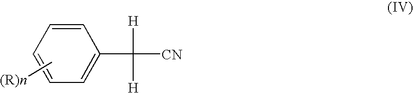
in which n and R are each as defined in the formula (I) with chlorine in the presence of catalytic amounts, or with a brominating agent, to the corresponding nitrile of the formula (II), which achieves direct coupling of the preparation of the nitrile of the formula (II) to the preparation of the corresponding alkyl 2,2-dichloro- or dibromophenylacetate of the formula (I).

6. A process for preparing alkyl 2,2-dichloro- or dibromophenylacetates of the formula (I), characterized in that, in a 1st stage, an optionally substituted benzyl cyanide of the formula 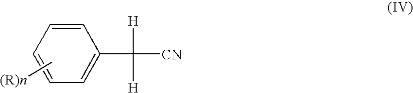
in which n may be an integer from 1 to 5 and R is hydrogen, CL-C₈-alkyl, aryl, heteroaryl, C₁-C₈-alkoxy, aryloxy or halogen, and R1 is C₁-C₈-alkyl, is reacted with chlorine in the presence of catalytic amounts of hydrogen chloride gas, or with a brominating agent, to give the corresponding nitrile of the formula 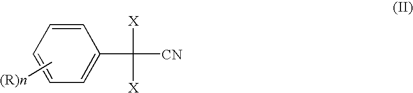
in which n and R are each as defined above and X is Cl or Br,
and the HCl or HBr offgas which forms is used in the second stage to convert the nitrile of the formula (II) to the corresponding alkyl 2,2-dichloro- or dibromophenylacetate of the formula 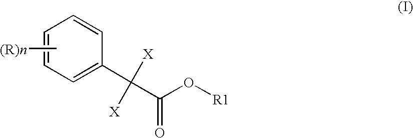
in which X, n and R are each as defined above and R1 is C₁-C₈-alkyl,
the conversion to the corresponding alkyl 2,2-dichloro- or dibromophenylacetate of the formula (I) in a second stage being effected in from 0.8 to 2 mol of water per mole of nitrile of the formula (II), from 1 to 8 mol of alcohol of the formula
R1OH (III)
in which R1 is as defined above, per mole of nitrile of the formula (II) and with from 1 to 3 mol of HCl or HBr in the form of the offgas from the 1st stage per mole of nitrile of the formula (II), optionally in the presence of a solvent inert under the reaction conditions, and the reaction temperature in the first phase of the conversion being from 30 to 60° C. and, in the second phase, from 60 to 100° C., whereupon, on completion of conversion, the reaction mixture is cooled to from 20 to 40° C. and diluted with water, and the corresponding alkyl 2,2-dichloro- or dibromophenylacetate of the formula (I) is isolated.

7. The process of claim 1, characterized in that the nitrile of the formula (II) is converted using an alcohol/water/HCl or HBr mixture which is obtained by passing HCl gas or HBr gas into a mixture of water and alcohol, or by passing HCl or HBr gas into a solution of alcohol and aqueous HCl or HBr, or by passing HCl or HBr gas into alcohol with subsequent dilution with water, and the desired molar ratio in the alcohol/water/HCl or HBr mixture can optionally be adjusted by diluting the aqueous, alcoholic HCl or HBr solution present with alcohol and/or water.

8. The process of claim 7, characterized in that the HCl gas or HBr gas used is an offgas, obtained according to claim 5, from the conversion of a benzyl cyanide of the formula (IV) to the corresponding nitrile of the formula (II), as a result of which the preparation of the nitrile of the formula (II) does not have to be coupled directly with the preparation of the alkyl 2,2-dichloro- or dibromophenylacetate of the formula (I), and HCl or HBr which are obtained as offgas in the preparation of the nitrile of the formula (II) may also be intermediately stored in the form of the alcohol/water/HCl or HBr mixture.

9. The process of claim 1, characterized in that the corresponding alkyl 2,2-dichloro- or dibromophenylacetates of the formula (I) are isolated by adding sufficient water that the precipitated ammonium chloride or bromide is just dissolved and a phase separation occurs, whereupon, optionally after extraction of the aqueous phase, water, alcohol and any extractant present are initially distilled out of the organic phase at atmospheric pressure and a maximum temperature of 90° C., and subsequently by-products under reduced pressure until a constant boiling temperature is attained, so that the corresponding alkyl 2,2-dichloro- or dibromophenylacetate of the formula (I) remains in the residue which may optionally be distilled overhead for further purification, or whereupon, optionally after extraction of the aqueous phase, the water is first removed from the organic phase on a water separator and alcohol and any extractant present are subsequently distilled off at atmospheric pressure.

10. The process of claim 9, characterized in that the crude alkyl 2,2-dichloro- or dibromophenylacetate of the formula (I), in the case that it contains too much organic acid, is admixed with an extractant from the group of hexane, heptane, toluene, ethers or esters and the corresponding alcohol of the formula (III) and again worked up distillatively.
